(19) United States
(12) Patent Application Publication
(10) Pub. No.: US 2009/0124888 A1
(43) Pub. Date: May 14, 2009

(54) Wing Design for RF Coil for MRI Imaging

(75) Inventors: Yiping Guan, Aurora, OH (US); Mark Richard, Cleveland, OH (US)

Correspondence Address: ULMER & BERNE, LLP;ATTN: DIANE BELL, 600 VINE STREET, SUITE 2800, CINCINNATI, OH, 45202-2409, US

(73) Assignee: Hitachi Medical Systems America, Inc., Twinsburg, OH (US)

(21) Appl. No.: 11/939,745

(22) Filed: Nov. 14, 2007

(51) Int. Cl. A61B 5/05

(52) U.S. Cl. 600/415

(57) ABSTRACT

The present invention relates to a housing for a radiofrequency receive coil for magnetic resonance imaging. The housing is designed for a coil wherein the patient lies on top of the coil in the prone position. The housing includes a base portion, a body portion and at least two wing portions. The base and body portions are generally constructed from a rigid material, while the wing portions are constructed from a flexible material. The wing portions may be connected using a connection strap, that applies tension to the wing portions to flex the wing portions in closer vicinity to the patient's body for optimal imaging.

### TECHNICAL FIELD

The invention relates generally to a design of the housing of a radiofrequency receive coil designed for magnetic resonance imaging of a patient's anatomy. More specifically, the invention relates to a design of a housing for a radiofrequency receive coil designed for a patient to lay in the prone position for magnetic resonance imaging.

### BACKGROUND OF THE INVENTION

Magnetic resonance imaging (MRI) is a medical diagnostic imaging technique used to diagnose many types of injuries and medical conditions. An MRI system includes a main magnet for generating a main magnetic field through an examination region. The main magnet is arranged such that its geometry defines the examination region. The orientation of the main magnet defines whether the MRI system is classified as a horizontal field system or a vertical field system. In a vertical field system, the static magnetic field is typically oriented in an anterior-posterior (A-P) direction relative to the prone/supine patient within the system. In a horizontal field system, the static main magnetic field is typically oriented in the head-foot (H-F) direction relative to the prone/supine patient within the system.

The main magnetic field causes the magnetic moments of a small majority of the various nuclei within the body to be aligned in a parallel or anti-parallel arrangement. The aligned magnetic moments rotate around the equilibrium axis with a frequency that is characteristic for the nuclei to be imaged. An external radiofrequency (RF) field applied by other hardware within the MRI system perturbs the magnetization from its equilibrium state. Upon termination of the application of the RF pulse, the magnetization relaxes to its initial state. During relaxation the time varying magnetic moment induces a detectable time varying voltage in the receive coil. The time varying voltage is commonly detected by a RF receive coil.

During operation of the RF receive coil, each element within the coil collects information from the time varying voltage induced by the magnetic moments within the anatomy of the patient nearest to that element. The information collected by each element is processed through the electronics within the MRI system on individual channels of the MRI system, which keep the information from each element separate throughout the imaging process. The information from each channel of the system is then processed by reconstruction software integrated with the MRI system to combine the single images from the channels to create a complete image of the anatomy of interest.

One or more RF receive coils, commonly called imaging coils, are typically placed within the vicinity of the patient during imaging. The imaging coil is typically comprised of a series of inductive and capacitive elements and operates by resonating and efficiently storing energy at what is known as the Larmor frequency. The imaging coil is comprised of at least one, and usually more than one element typically made of a continuous piece of copper in a solenoid, loop, butterfly or figure-eight (saddle), or other continuous geometric shape. The elements are positioned at various locations throughout coil to provide for the desired imaging of the patient. The design of the receive coil varies depending on whether it is designed for use within a vertical or horizontal field MRI system.

The shape, configuration and location of elements within the receive coil affect the characteristics of the coil, including the coil sensitivity, signal-to-noise ratio (SNR) and imaging field-of-view. Conventionally, the receive coil's imaging field-of-view (FoV) is defined as the distance between the two points on the coil sensitivity profile, which is a graph of the coil's sensitivity over the distance profile, where the signal drops to 80% of its peak value. The shape and design of the RF receive coil varies depending on the patient anatomy the coil is designed to imagine.

Further developments in MRI include various parallel imaging techniques. An example of a parallel imaging technique is Simultaneous Acquisition of Spatial Harmonics (SMASH). The SMASH technique uses a parallel processing algorithm to exploit spatial information inherent in a surface coil array. The result is an increase in MR image acquisition speed, resolution and/or field of view. In a similar fashion, another parallel processing algorithm is known where the acceleration of image acquisition is performed on the time domain space instead of the frequency domain space. This parallel acquisition technique is referred to as Sensitivity Encoding (SENSE). In SENSE, images are obtained by means of magnetic resonance (MR) of an object placed in a static magnetic field and includes simultaneous measurement of a number of sets of MR signals by application gradients and an array of receiver coils. The characteristics of all of these parallel imaging techniques is that the acceleration speed is directly proportional to the number of independent receivers along the direction that the image acceleration needs to be applied. Thus, the higher the number of receiver coils, the faster the acceleration speed for acquiring an image with better SNR and improved image quality.

Certain RF coils, such as coils designed to image the breast region of a patient, are designed for the patient to be positioned laying in a prone position on top of the RF coil, Coils designed for the patient to lie in the prone position on top of the coil are available for MRI systems having a vertical or a horizontal magnetic field. Coils of the prior art designed for the patient to lie in the prone position have been designed using stiff and non-flexible materials, so as to provide the shape necessary to allow for high quality images of the region of interest, generally the patient's breasts and chest tissues. In using breast coils of the prior art, the patient generally lies on top of the breast coil, with their breasts positioned within concave cut-outs (commonly called cups) in the coil. The coil is generally made of non-flexible material so the coil can adequately support the weight of the patient's body, while maintaining the shape of the coil to provide high quality images of the patient's breasts.

It has become known that it is sometimes necessary to image the tissues of the patient's chest wall, as well as the underarm region, along with the tissues of the patient's breasts. The coils of prior art are commonly designed to provide high quality images of the patient's breasts, however the image quality decreases as the distance of the tissue increases from the patient's breasts. So, for example, when the tissues of the side of the patient's chest wall or underarm regions are of interest, the coils of prior art produce images of decreased quality than the images of the patient's actual breast tissue.

### SUMMARY OF THE INVENTION

The housing design of the present invention seeks to remedy some of the problems described above. The housing design of the present invention provides for a housing for a radiofrequency (RF) receive coil (also commonly referred to as an imaging coil) designed to image a patient's breast region, including the chest wall, underarm and side regions. The housing of the present invention is intended for implementation with any coil that is designed for the patient to lie in a prone on top of the coil for magnetic resonance imaging (MRI) in at least certain imaging situations. Examples of coils in which the present housing invention may be useful include, but are not limited to, coils designed for the imaging of the patient's breast region, torso, back and abdomen. The housing of the present invention may be utilized in the design of RF coils for use with a vertical or a horizontal magnetic field.

The housing design of the present invention provides for a housing that is constructed from differing materials, wherein the housing adequately supports the weight of the patient on top of the coil, keeping the general shape of the coil beneath the patient, while providing flexibility in the material on the sides of the coil housing, so the coil can be flexed and wrapped to encompass at least a portion of the patient's side regions.

Generally, the design of the breast coil housing described herein includes a base portion of the coil, a body portion of the coil, and wing portions of the coil. The base portion of the coil may or may not be integrated with the body portion of the coil. The base portion of the coil is designed to rest upon the table of the MRI system. The body of the coil rises above the table of the MRI system and generally includes two concave portions for the acceptance of the patient's breasts, referred to throughout this application as the cups of the coil. The patient's chest wall is supported by portions of the body of the coil that are designed to come in contact with the patient's chest wall as the patient lies on top of the coil in the prone position. At least two wing portions exist on the coil, with at least one wing portion on the left side of the coil and at least one wing portion on the right side of the coil. It is foreseen that more than one wing portion can exist on either side of the coil, depending on the element design used within the coil. The wing portions will be created using a flexible material that allows the wing portions to be flexed around a portion of the patient's torso, and will generally be wrapped partially up the patient's side region. The wing portions may be secured using a strap that connects around the patient's back region.

The housing protects the coil elements within the coil. The quantity and type of the elements as well as the positioning of the elements within a coil using the present housing design may vary depending on the exact design of the coil. Commonly, elements will be positioned in the vicinity of the coil cups, and within the body of the coil housing. Further elements will exist within the wing portions of the coil. The elements that exist within the wing portions of the coil will be essential in producing high quality images of the patient's side and underarm regions and associated tissues. This invention provides no limitation as to the type of elements that may be positioned within the coil, and especially within the wing portion of the housing. Any geometry element known in the art may be positioned within the wing portion of the housing, as is beneficial to the design of the specific coil the housing of the invention is being used with. The shape of the wing portion may be uniquely designed to incorporate whatever geometry elements will exist within the wing portion of the housing, depending on the electrical design of the coil.

It is an object of this invention to provide a design for a housing for a RF receive coil for MR imaging wherein the patient lies in a prone position on top of the RF receive coil, such as when imaging the patient's breast region.

These and other objects of the present invention will become more readily apparent from a reading of the following detailed description taken in conjunction with the accompanying drawings wherein like reference numerals indicate similar parts, and with further reference to the appended claims.

### BRIEF DESCRIPTION OF THE DRAWINGS

The preferred embodiment of the invention, illustrative of the best mode in which applicant has contemplated applying the principals is set forth in the following description and is shown in the drawings and is particularly and distinctly pointed out and set forth in the appended claims. The invention may take physical form in certain parts and arrangements of parts, numerous embodiments of which will be described in detail in the specification and illustrated in the accompanying drawings which form a part hereof, and wherein:

FIG. 1 is a side perspective view of a coil having the housing of an embodiment of the present invention;

FIG. 2 is a side perspective view of a coil having the housing of an embodiment of the present invention;

FIG. 3 is a top perspective view of a coil having the housing of an embodiment of the present invention; and

FIG. 4 is a side perspective view of a patient positioned on a coil having the housing of an embodiment of the present invention.

### DETAILED DESCRIPTION OF THE INVENTION

Referring now to the drawings wherein the showings are for purposes of illustrating numerous embodiments of the invention only and not for purposes of limiting the same, the figures illustrate the novel idea of a housing design for a radiofrequency (RF) receive coil (also commonly referred to as an imaging coil) designed such that the patient is positioned in a prone position on top of the coil (and the housing surrounding the coil). The housing of the present invention is intended for implementation with any coil that is designed for the patient to lie in a prone on top of the coil for magnetic resonance imaging (MRI) in at least certain imaging situations. The housing is specifically designed for a coil meant to image a patient's breast region, including the chest wall, underarm and side regions. Examples of coils in which the present housing invention may be useful include, but are not limited to, coils designed for the imaging of the patient's breast region, torso, back and abdomen. The housing of the present invention may be utilized in the design of RF coils for use with a vertical or a horizontal magnetic field, including systems with a closed or open bore design, of any magnetic strength.

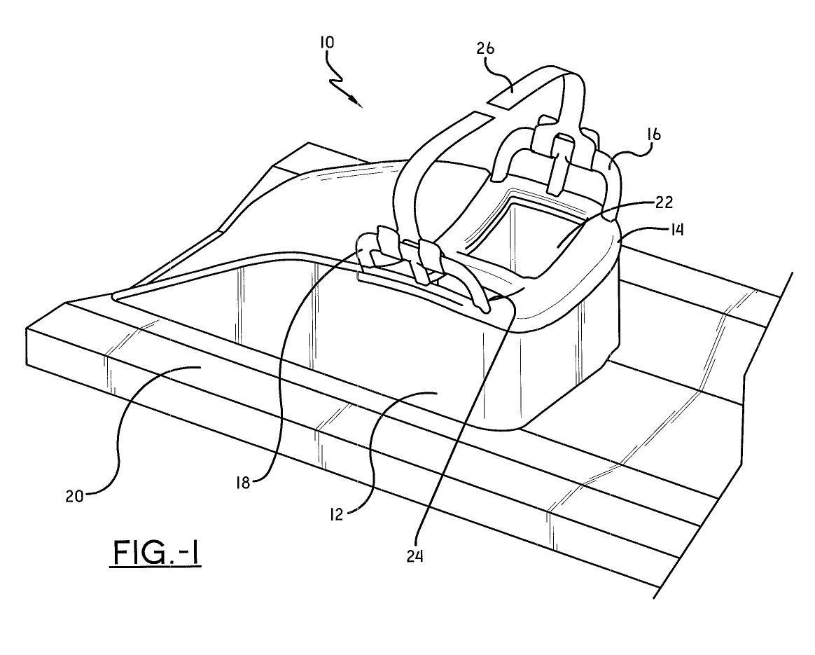

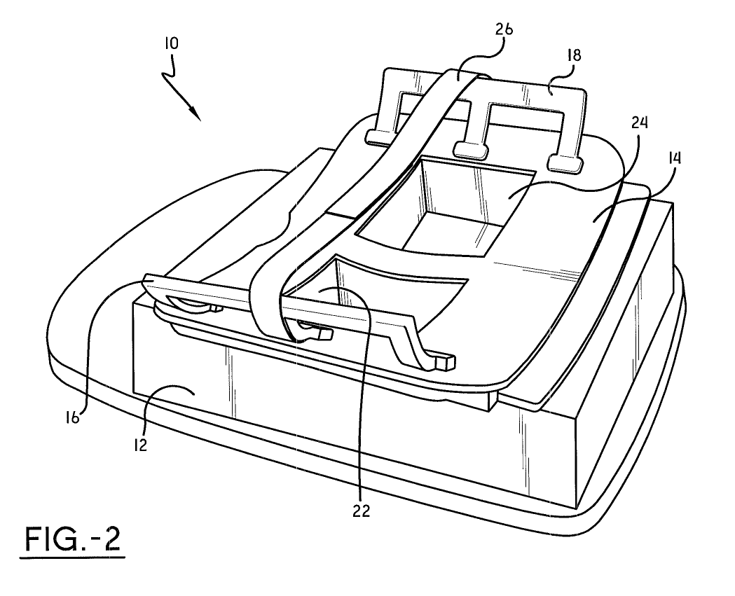

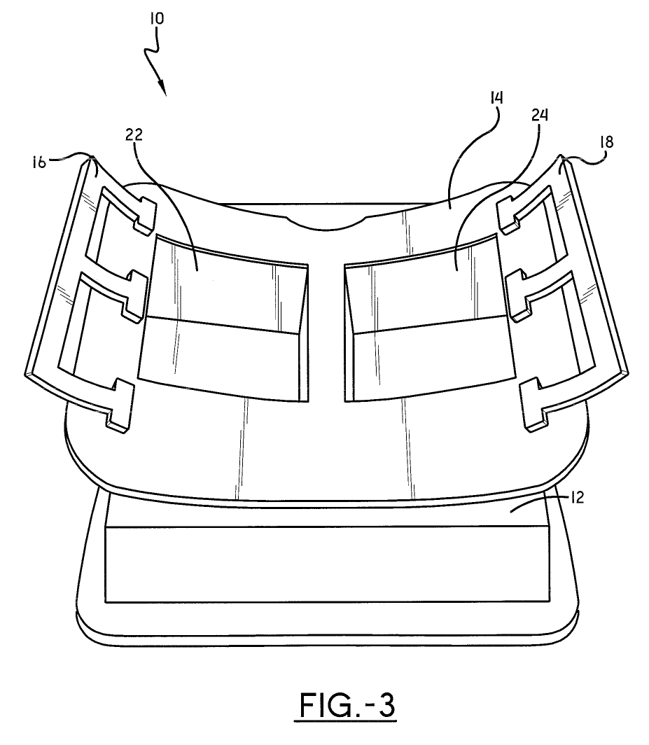

The housing design of the present invention, embodiments of which are illustrated in FIGS. 1-3, provides for a housing for a RF coil that is constructed from differing materials, wherein housing 10 adequately supports the weight of the patient on top of the coil, keeping the general shape of the coil beneath the patient, while providing flexibility in the material on the sides of the coil housing, so the sides of the coil can be flexed and wrapped to encompass at least a portion of the patient's side or underarm regions. Generally, the design of breast coil housing 10 described herein includes base portion 12 of the coil, body portion 14 of the coil, and wing portions 16 and 18 of housing 10. Base portion 12 of housing 10 may be integrated with body portion 14 of housing 10 in some embodiments, however may be a separate portion and not integrated with body portion 14 of housing 10 in further embodiments of housing 10. Base portion 12 of housing 10 is designed to rest upon table 20 of the MRI system. Body portion 14 of housing 10 rises above table 20 of the MRI system and generally includes two concave portions for the acceptance of the patient's breasts, referred to throughout this application as coil cups 22 and 24. The patient's chest wall is supported by sections of body portion 14 of housing 10 that are designed to come in contact with the patient's chest wall as the patient lies in the prone position on top of the coil defined by housing 10. These sections of the housing designed to support the patient commonly contact the patient at the breastbone, between the patient's breasts, and may also contact the patient at the bottom of the ribcage, against the upper abdomen region or beneath the patient's breasts.

At least two wing portions 16 and 18 exist on the coil, with at least one wing portion 16 on the left side of the coil and at least one wing portion 18 on the right side of the coil. It is foreseen that more than one wing portion can exist on either side of the housing, depending on the element design used within the coil. Certain embodiments of the invention provide that wing portions 16 and 18 will be created using a flexible material that allows wing portions 16 and 18 to be flexed around a portion of the patient's torso, and will generally be wrapped partially up the patient's side region. It is also foreseen that further embodiments may include wing portions made of a rigid material, where a flexible material is provided connecting the wing portions to the body portion of the housing, allowing the rigid wing portions to be flexed over the patient's torso. The wing portions of the invention may be secured using connection strap 26 that connects around the patient's back region.

Connection strap 26 will generally attach to both wing portions 16 and 18 of housing 10, and will be of any design adequate in securing connection strap 26, providing tension on wing portions 16 and 18 to bring them in a closer vicinity to the patient's body. Connection strap 26 may adhere to itself, such that a half of connection strap 26 is attached to left wing portion 16 and a half of connection strap 26 is attached to wing portion 18, with the two halves of connections trap 26 connecting to one another. In further embodiments the connection strap may be wholly secured to just one of the wing portions, with the connection strap being connected to the other wing portion. The connection strap may any connection means, including buttons, snaps, hooks, clasps, or preferably a hook and loop device such as Velcro or other type of removable connection. As previously described, the connection strap may also be self-adhering, connecting to itself. The connection strap will commonly be adjustable in length, to accommodate different sized patients. The connection strap will generally be constructed from a fabric, nylon, or plastic material, however the material of the connection strap or the connector pieces is limited only by the requirement that the material not affect the images created by the RF coil having the housing design of the present invention, including the connection strap. The connection strap may also be made using a material having elastic properties, to provide a connection strap that can be quickly and easily adjusted to the size of the patient.

Housing 10 of the present invention generally protects the coil elements within the coil. The quantity and type of the elements as well as the positioning of the elements within a coil using the present housing design may vary depending on the exact design of the coil. Commonly, elements will be positioned in the vicinity of the coil cups, and within the body of the coil housing. Further elements will exist within the wing portions of the housing. The elements that exist within the wing portions of the housing will be essential in producing high quality images of the patient's side and underarm regions and associated tissues. This invention provides no limitation as to the type of elements that may be positioned within the coil, and especially within the wing portion of the housing. Any geometry element known in the art may be positioned within the wing portion of the housing, as is beneficial to the design of the specific coil the housing of the invention is being used with. The shape of the wing portion may be uniquely designed to incorporate whatever geometry elements will exist within the wing portion of the housing, depending on the electrical design of the coil.

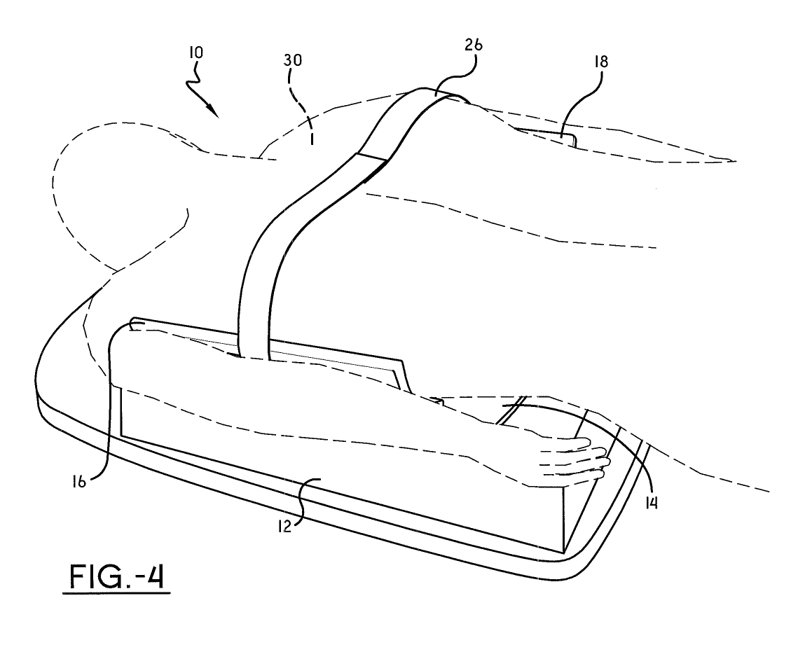

Proper positioning of the breast coil using housing 10 of the present invention on the breast region of patient 30 is illustrated in FIG. 4. When the coil is properly positioned on a patient's anatomy for MR imaging of breast region using, patient 30 lies face down on top of the coil in the prone position. The patient's breasts are positioned within coil cups 22 and 24 (as illustrated in FIGS. 1-3, hidden by patient 30 in FIG. 4), and are ideally, but not necessarily, centered within. There may be distance between the walls of coil cups 22 and 24 and the patient's breasts, or they may fit snugly, depending on the size of the patient's anatomy and the size of coil cups 22 and 24. Patient 30 is supported by the portions of coil housing 10 that are designed to contact with the patient's chest wall. Certain embodiments of the coil may provide a head rest for the patient to rest their head on, or standard or specially designed pillows may be provided for the patient's head.

The design of the coil housing of the present invention offers many advantages over past designs. The wing portions of the housing allow for optimal imaging of the patient's side region, underarm region, and associated tissues. The flexibility of the wing portions allows for the housing to provide a unique fit to each individual patient imaged within a coil using the housing of the present invention. The positioning of the flexible wings is not completely limited by the other dimensions of the coil, as is the case with previous flexible coils, for example the positioning of previous flexible body coils of the prior art are limited by the diameter of the coil. The present flexible wing design also improves patient comfort, removing rigid coil portions from presenting discomfort to the patient's side and underarm regions.

The housing of the present invention is designed to protect the elements used within the RF coil having the housing of the present invention. The elements may be, but are not necessarily, mounted on a frame structure to support the elements and ensure the elements are not bent or repositioned during imaging use of the coil. In coils using a frame structure, the housing of the present invention will also protect the frame structure along with the elements within the coil. The RF coil having the housing of the present invention may also include electronic components in electrical connection with the elements of the design. These electronic components transmit the signal generated within the coil to the other components of the MR system to create the desired images of the patient. The housing of the present invention may also house these electrical components.

The housing structure of the present invention is constructed of a combination of materials, using a rigid material, such as a hard plastic or polymer for the base and body portions of the housing, and using a flexible material, such as a foam, rubberized polymer, or other similar material for the wing portions of the housing. The housing will be of any material that can adequately protect the internal elements and associated electronics of the coil from any external pollutants such as moisture, dust and the like, with the only limitation being that the material used to construct the housing must not affect the MR images created by the coil.

The base and body portions, and wing portions of the housing may each be created from more than one type of material. For example, the wing portions of the housing may be constructed from a flexible foam, and also covered in a second material such as a vinyl or plastic that allows for easy cleaning of the coil. Furthermore, a different rigid material may be used to house the more sensitive electronics of the coil than is used in construction of the body portion of the coil housing. It is also foreseen that the electronic components other than the elements may be located in the same housing as the coil elements, below the coil elements, between the MRI table and the elements, or elsewhere in the coil. In most embodiments the housing structure will be shaped to conform with not only the element design of the coil, but also to the general shape of the patient's chest and breast region to provide a comfortable fit during imaging of the patient.

In a preferred embodiment of the design of this invention, the coil of this design can be used to image a significant majority of patients' breast and chest regions, and does not require different sized coils to obtain optimal imaging of patients. It is foreseen however, that a larger or smaller size coil may be created using the design of this invention to provide optimal imaging for very large or very small patients. The size and shape of the wing portions of the housing may be increased or decreased, depending on the element design of the coil and further depending on the size of the patient the coil is designed to accept. The overall length of the connection strap may be increased or decreased to affect the span and tightness the wing portions may provide coverage for.

Described herein is a housing for a magnetic resonance imaging radiofrequency receive coil for imaging the human breast region of a patient, wherein at least one element exists within the housing, the housing including a base portion, wherein the base portion is shaped to rest on a table of a magnetic resonance imaging machine; a body portion, wherein the body portion includes two openings sized for the acceptance of a patient's breasts; and at least two wing portions, wherein at least one of the at least two wing portions is connected to a left side of the body portion, and at least one of the at least two wing portions is connected to a right side of the body portion, and further wherein the at least two wing portions flexibly extend above the body portion of the housing and connect over the patient positioned within the radiofrequency receive coil. In embodiments of the housing the base portion and the body portion of the housing are integrated. In other embodiments of the housing more than one wing portion exists on the right side of the body portion of the housing and more than one wing portion exists on the left side of the body portion of the housing. In another embodiment of the invention the at least two wing portions connect using a connection strap. In certain other embodiments the connection strap is constructed of a material having elasticity. In embodiments of the housing the at least two wings are constructed of a flexible material, and in other embodiments the at least two wings are constructed of a rigid material and are connected to the body portion of the housing a flexible material.

Further described herein is a housing for a magnetic resonance imaging radiofrequency receive coil for imaging a patient, wherein at least one element exists within the housing, the housing including a base portion, wherein the base portion is shaped to rest on a table of a magnetic resonance imaging machine; body portion, wherein the body portion extends above the base portion; and at least two wing portions, wherein at least one of the at least two wing portions is connected to a left side of the body portion, and at least one of the at least two wing portions is connected to a right side of the body portion, and further wherein the at least two wing portions flexibly extend above the body portion of the housing. In certain embodiments of the housing the base portion and the body portion are integrated. In other embodiments more than one wing portion exists on the right side of the body portion of the housing and more than one wing portion exists on the left side of the body portion of the housing. In certain embodiments of the housing the at least two wing portions connect using a connection strap, and in other embodiments the connection strap is constructed of a material having elasticity. In certain embodiments of the housing the at least two wings are constructed of a flexible material, and in other embodiments of the housing the at least two wings are constructed of a rigid material and are connected to the body portion of the housing a flexible material.

Also taught within is a magnetic resonance imaging radiofrequency receive coil having at least one element protected by a housing, the housing of the coil including a base portion, wherein the base portion is shaped to rest on a table of a magnetic resonance imaging machine; a body portion, wherein the body portion includes at least one support portion for supporting the weight of a patient in a prone position being imaged by the coil; and at least two wing portions, wherein at least one of the at least two wing portions is connected to a left side of the body portion, and at least one of the at least two wing portions is connected to a right side of the body portion, and further wherein the at least two wing portions flexibly extend above the body portion of the housing. In certain embodiments of the coil the base portion and the body portion of the housing are integrated. In other embodiments of the coil more than one wing portion exists on the right side of the body portion of the housing and more than one wing portion exists on the left side of the body portion of the housing. In other embodiments of the coil the at least two wing portions connect over the patient using a connection strap, which in certain embodiments is constructed of a material having elasticity. In embodiments of the coil the at least two wings are constructed of a flexible material, and in other embodiments of the coil the at least two wings are constructed of a rigid material and are connected to the body portion of the housing a flexible material.

In the foregoing description, certain terms have been used for brevity, clearness, illustration and understanding; but no unnecessary limitations are to be implied therefrom beyond the requirements of the prior art, because such terms are used for descriptive purposes and are intended to be broadly construed. Moreover, this invention has been described in detail with reference to specific embodiments thereof, including the respective best modes for carrying out each embodiment. It shall be understood that these illustrations are by way of example and not by way of limitation.

What is claimed is:

1. A housing for a magnetic resonance imaging radiofrequency receive coil for imaging the human breast region of a patient, wherein at least one element exists within said housing, said housing comprising:
(a) a base portion, wherein said base portion is shaped to rest on a table of a magnetic resonance imaging machine;
(b) a body portion, wherein said body portion includes two openings sized for the acceptance of a patient's breasts; and
(c) at least two wing portions, wherein at least one of said at least two wing portions is connected to a left side of said body portion, and at least one of said at least two wing portions is connected to a right side of said body portion, and further wherein said at least two wing portions flexibly extend above said body portion of said housing and connect over said patient positioned within said radiofrequency receive coil.

2. The housing of claim 1 wherein said base portion and said body portion of said housing are integrated.

3. The housing of claim 1 wherein more than one wing portion exists on said right side of said body portion of said housing and more than one wing portion exists on said left side of said body portion of said housing.

4. The housing of claim 1 wherein said at least two wing portions connect using a connection strap.

5. The housing of claim 4 wherein said connection strap is constructed of a material having elasticity.

6. The housing of claim 1 wherein said at least two wings are constructed of a flexible material.

7. The housing of claim 1 wherein said at least two wings are constructed of a rigid material and are connected to said body portion of said housing a flexible material.

8. A housing for a magnetic resonance imaging radiofrequency receive coil for imaging a patient, wherein at least one element exists within said housing, said housing comprising:
(a) a base portion, wherein said base portion is shaped to rest on a table of a magnetic resonance imaging machine;
(b) a body portion, wherein said body portion extends above said base portion; and
(c) at least two wing portions, wherein at least one of said at least two wing portions is connected to a left side of said body portion, and at least one of said at least two wing portions is connected to a right side of said body portion, and further wherein said at least two wing portions flexibly extend above said body portion of said housing.

9. The housing of claim 8 wherein said base portion and said body portion of said housing are integrated.

10. The housing of claim 8 wherein more than one wing portion exists on said right side of said body portion of said housing and more than one wing portion exists on said left side of said body portion of said housing.

11. The housing of claim 8 wherein said at least two wing portions connect using a connection strap.

12. The housing of claim 11 wherein said connection strap is constructed of a material having elasticity.

13. The housing of claim 8 wherein said at least two wings are constructed of a flexible material.

14. The housing of claim 8 wherein said at least two wings are constructed of a rigid material and are connected to said body portion of said housing a flexible material.

15. A magnetic resonance imaging radiofrequency receive coil having at least one element protected by a housing, said housing of said coil comprising:
(a) a base portion, wherein said base portion is shaped to rest on a table of a magnetic resonance imaging machine;
(b) a body portion, wherein said body portion includes at least one support portion for supporting the weight of a patient in a prone position being imaged by said coil; and
(c) at least two wing portions, wherein at least one of said at least two wing portions is connected to a left side of said body portion, and at least one of said at least two wing portions is connected to a right side of said body portion, and further wherein said at least two wing portions flexibly extend above said body portion of said housing.

16. The coil of claim 15 wherein said base portion and said body portion of said housing are integrated.

17. The coil of claim 15 wherein more than one wing portion exists on said right side of said body portion of said housing and more than one wing portion exists on said left side of said body portion of said housing.

18. The coil of claim 15 wherein said at least two wing portions connect over said patient using a connection strap.

19. The coil of claim 15 wherein said connection strap is constructed of a material having elasticity.

20. The coil of claim 15 wherein said at least two wings are constructed of a flexible material.

21. The coil of claim 15 wherein said at least two wings are constructed of a rigid material and are connected to said body portion of said housing a flexible material.
